(19) United States
(12) Patent Application Publication
(10) Pub. No.: US 2026/0174953 A1
(43) Pub. Date: Jun. 25, 2026

(54) APPLICATOR DEVICE FOR INSULIN PATCH PUMP WITH CANNULA DEPTH CONTROL

(71) Applicant: Tandem Diabetes Care Switzerland Sàrl, St-Sulpice (CH)

(72) Inventors: Adrian Ramseyer, St-Sulpice (CH); Vincent Vaucher, St-Sulpice (CH)

(21) Appl. No.: 19/419,869

(22) Filed: Dec. 15, 2025

Related U.S. Application Data
(60) Provisional application No. 63/737,021, filed on Dec. 20, 2024.

(51) Int. Cl. A61M 5/142; A61M 5/42; A61M 5/46

(52) U.S. Cl. CPC A61M 5/14248; A61M 2005/14252; A61M 5/425; A61M 5/46; A61M 2210/04

(57) ABSTRACT

Embodiments disclosed herein are directed to an applicator for inserting a cannula into a body of the user for delivering medication from a medication infusion device to the user. According to one aspect, an applicator device includes a cannula and an insertion needle configured to insert the cannula into a body of a user. The applicator can include features that control the geometry of the skin during insertion of the cannula to provide a more reliable depth and insertion angle for the cannula.

### PRIORITY CLAIM

The present application claims the benefit of U.S. Provisional Patent Application No. 63/737,021 filed Dec. 20, 2024, which is hereby incorporated by reference herein in its entirety.

### TECHNICAL FIELD

The present disclosure relates generally to ambulatory infusion pumps and, more particularly, to applicators for attaching ambulatory infusion pumps to user's body.

### BACKGROUND

Wearable insulin pumps are known for providing a Type I Diabetes Mellitus patient with small doses of short acting insulin continuously (basal rate). The devices also can be used to deliver variable amounts of insulin when a meal is consumed (bolus). The basal insulin rates are usually programmed in a pump by a physician, and one or multiple basal settings may be programmed in the pump based on the patient's needs. The patient may program the amount of insulin for a mealtime bolus directly on the pump. Most pumps also include bolus calculators to help the patient determine the amount of insulin the patient may need at mealtime based on the patient's glucose levels and the amount of carbohydrates the patient may consume. The objective is to control the patient's blood glucose level within a desired range. Some such insulin pumps are coupled to an adhesive patch that permits the pump to be directly adhered to a user's body surface, for example the abdomen, and are referred to as “patch pumps.” In addition, some previously known systems were configured to interface wirelessly with a continuous glucose monitor, which typically also may be disposed on a patch designed to be adhered to the user's body. Other previously known systems employ still further modules designed to monitor user activity and report that activity to a controller associated with the patch pump to titrate the insulin delivery in accordance with the user's activity level.

For patch pumps, a transcutaneous cannula generally needs to be inserted into the skin for delivering medication transcutaneously from an external fluid reservoir associated with the patch pump that is in fluid communication with the cannula. Thus, the cannula needs to be inserted into the skin before delivering the medication. Patch pump systems may include an applicator that can simultaneously insert the cannula into the user's skin and affix an adhesive pad onto the user's skin that holds the patch pump.

### SUMMARY

Embodiments disclosed herein are directed to an applicator for inserting a cannula into a body of the user for delivering medication from a medication infusion device to the user.

According to one aspect, an applicator device includes a cannula and an insertion needle configured to insert the cannula into a body of a user. The applicator can include features that control the geometry of the skin during insertion of the cannula to provide a more reliable depth and insertion angle for the cannula.

In an embodiment, an applicator for inserting a cannula into a body of a patient can include a cannula and an insertion needle having a sharpened distal tip to insert the cannula into the body of the patient. An actuation assembly can be configured to actuate the insertion needle to insert the cannula. A skin deformation projection can be configured to deform the skin in an area where the cannula is inserted.

In an embodiment, an applicator extension for use with an applicator for inserting a cannula into a body of a patient can include a body having an opening configured to receive an applicator housing of the applicator therein and a skin deformation projection extending from the body configured to deform skin of a patient in an area where the cannula is inserted.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is an exemplary medication infusion system according to the disclosure.

FIG. 2 is a diagram showing exemplary attachment zones for the patch pump of FIG. 1 and an external sensor.

FIGS. 3A and 3B are, respectively, a perspective view and an exploded view of the pad and applicator of FIG. 1.

FIGS. 4A-4B depict an exemplary cannula for delivering medication transcutaneously.

FIG. 5 schematically depicts an applicator for a patch pump.

FIGS. 6A-6E schematically depict use of an applicator for a patch pump.

FIGS. 7A-7E schematically depict use of an applicator for a patch pump.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

The following detailed description should be read with reference to the drawings in which similar elements in different drawings are numbered the same. The drawings, which are not necessarily to scale, depict illustrative embodiments and are not intended to limit the scope of the invention.

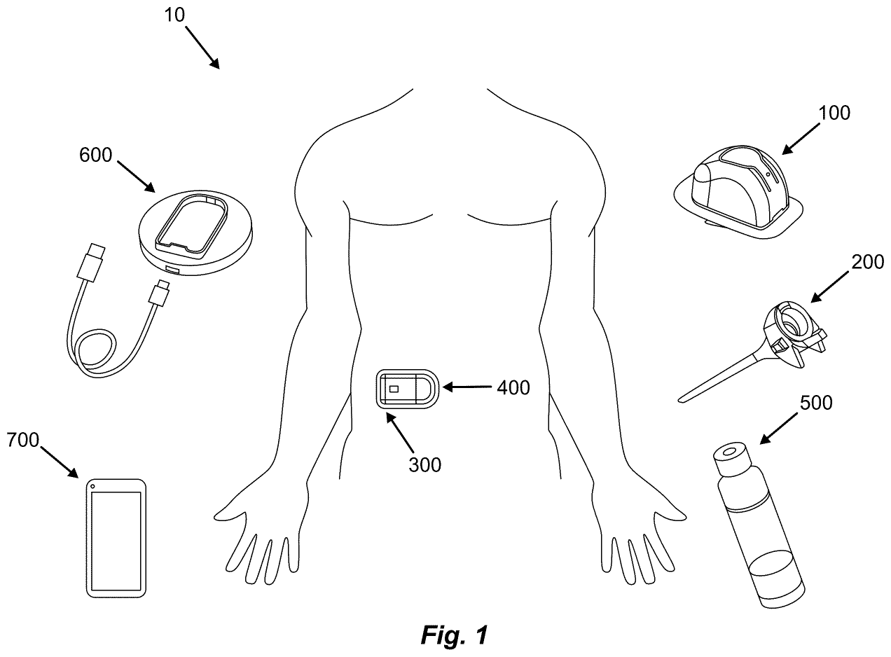

Referring to FIG. 1, an exemplary medication infusion system including a patch pump for delivering medication is described. In FIG. 1, components of the system are not depicted to scale on either a relative or absolute basis. Medication infusion system 10 can include applicator 100, cannula 200, pump 300, cap 400, cartridge 500, charging system 600 and/or software application 700. Preferably, applicator 100, cannula 200, cap 400, and cartridge 500 are disposable components that may be replaced approximately every 3-10 days and/or once the pre-filled cartridge is empty, while pump 300 is reusable and may last for an extended period of time, e.g., approximately 2-4 years. As such, pump 300 may be used with many different applicators, cannulas, caps, and pre-filled cartridges. Such a configuration is expected to promote sanitary use of the system, as the components exposed to the patient and the insulin are disposable, while reducing costs for components containing more expensive electronics, e.g., pump 300, charging system 600, and/or software application 700, which may be used repeatedly. In some embodiments, system 10 includes a second pump, such that the wearer may charge the second pump while using the first pump and vice versa. In this manner, the wearer will always have a pump that is charged and ready to be used once the cartridge of the pump in use is empty. Further, this system can be designed to reduce waste while reducing the number of times the wearer is required to insert a new cannula. Medication infusion system 10 may be used to apply cannula 200 and a pad to a wearer and to deliver medication through cannula 200 via a patch pump coupled to the pad. Further details regarding such a system can be found in U.S. Patent Publication No. 2022/0379014, which is hereby incorporated herein by reference in its entirety.

Applicator 100 is configured to apply an adhesive pad to the wearer and, upon actuation, to insert cannula 200 into the wearer. The pad is configured to be secured to the wearer for a period of time, e.g., at least 3 days, 7-10 days, etc., and then may be replaced by a similar pad using a similar applicator. The pad may include a pad skeleton having one or more locking mechanisms that are configured to couple the pad to applicator 100 for insertion of cannula 200 or to the assembled pump for delivery of medication. Applicator 100 may include an internal component configured to support an insertion mechanism designed to insert cannula 200 through the skin of the wearer via rotational movement and to guide and orient cannula 200 during insertion.

Preferably, applicator 100 is designed to suppress noise during insertion. The insertion mechanism may include an applicator needle configured to pierce the wearer's skin and a biasing member, which may be coupled to one or more links configured to interact with cannula 200 and the applicator needle. Upon actuation by the wearer, the insertion mechanism preferably rotates and applies a distal force on cannula 200 and the applicator needle within cannula 200, such that cannula 200 is inserted through the wearer's skin. Cannula 200 may include a proximal cannula head configured to couple to one or more locking mechanisms on the pad skeleton and, at the same time, uncouple applicator 100 from the pad skeleton. The insertion mechanism further may be configured to continue rotating to withdraw the applicator needle from cannula 200 and to store the applicator needle within the applicator after cannula 200 is inserted.

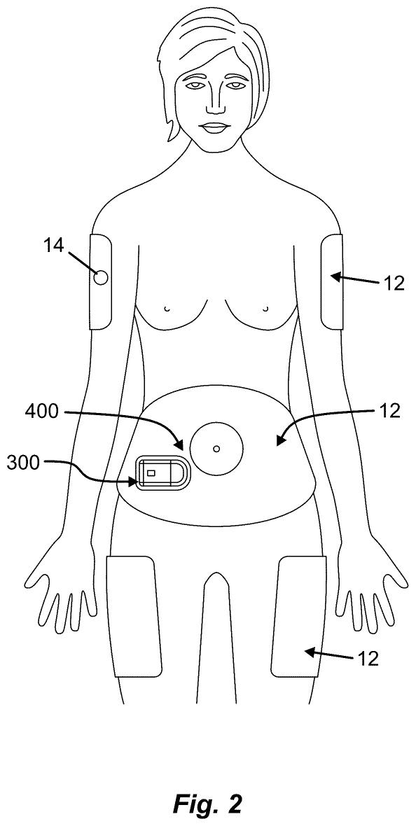

Referring now to FIG. 2, exemplary attachment zones for the patch pump and an optional external sensor, such as a continuous glucose monitoring sensor are illustrated. Attachment zones 12 illustrate several locations on the wearer's body where the applicator may attach the adhesive pad and insert the cannula and to which the patch pump is secured. For example, the patch pump may be secured to the upper arms, abdomen, or thighs of the wearer. As will also be understood by one of ordinary skill in the art, the patch pump may be secured to other locations on the wearer.

The patch pump also may be operatively coupled to an optional continuous glucose monitoring sensor, which may transmit data to a controller of the patch pump, which data may be used to adjust the time of insulin delivery or the amount of each dose. Preferably, the patch pump receives data from continuous glucose monitoring sensor 14, which is configured to be attached within attachment zones 12.

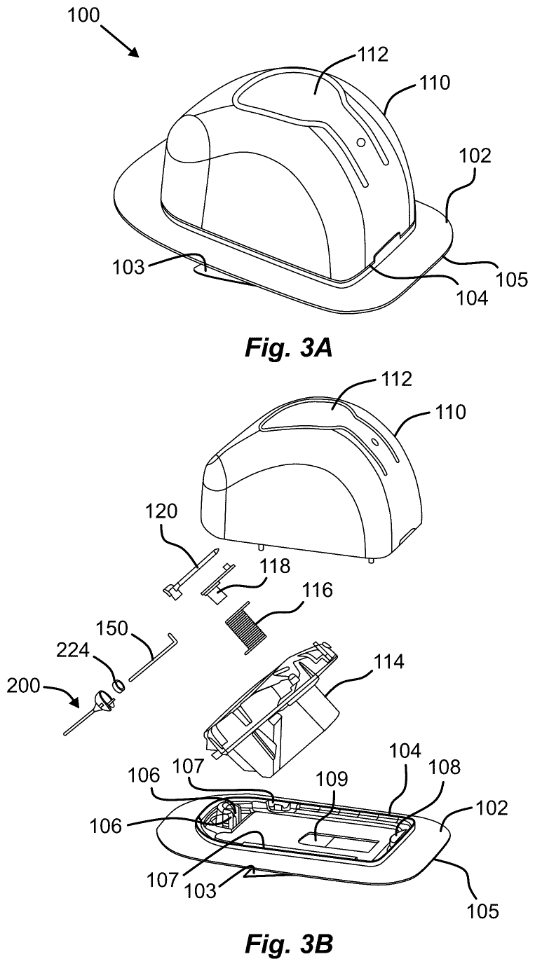

Referring now to FIGS. 3A and 3B, perspective and exploded views of an exemplary pad and applicator are described. Applicator 100 may transcutaneously apply a cannula, upon actuation by a user, which is designed to deliver doses of medication (e.g., insulin) from a patch pump configured to be removable coupled to the cannula. Advantageously, applicator further may apply a pad that is adhered to the wearer's skin and then coupled to the patch pump. For example, actuation of applicator 100 may both insert the cannula and cause the cannula to be locked to the adhesive pad in a single actuation. Further, applicator 100 may include internal components designed to minimize noise during the actuation process. For example, applicator 100 may avoid clicks and/or hard stops that make audible noises during insertion of the cannula.

In a pre-actuation state, applicator 100 may be coupled to pad 102 as shown in FIG. 3A. For example, applicator 100 may be coupled to pad 102 via pad skeleton 104 of pad, which is disposed on a first surface of pad 102. Skin-safe pad adhesive 105 may be disposed on a second, skin-facing surface of pad 102 such that the pump-pad assembly may be attached to a wearer for a period of time, for example, 3-5 days, 3-10 days, or 10 days or more. One or more release liners 103 may be attached to pad adhesive 105 until pad 102 is ready to be secured to the wearer. Pad skeleton 104 may be a frame with a shape designed to surround the pump-cap assembly so as to securely couple the adhesive pad to the pump-cap for wearing by the patient. Pad skeleton 104 may be designed to removably couple portions of pad 102 to applicator 100 in the pre-actuation state. For example, pad skeleton 104 may have one or more attachment mechanisms to lock pad 102 to applicator 100 and unlock upon actuation of applicator 100. Advantageously, the attachment mechanisms also may lock the cannula to pad 102 after actuation. As depicted in FIG. 3A, pad skeleton 104 may have pad attachments 106 at a first end of pad 102 and pad back clip 108 at a second end of pad 102. Pad attachments 106 and pad back clip 108 may interact with applicator 100 or a patch pump to lock the pad to applicator 100 or the patch pump. Pad attachments 106 may include at least two arms that protrude upwards from the pad and away from the skin surface of the wearer. Each arm may have an opening (e.g., slot) to receive extensions from the applicator during pre-actuation and extensions from the cannula post-actuation. Thus, the arms, which may have a U-shape, and openings may be used to lock to both the applicator and the cannula. Pad skeleton 104 may also include pad clips holes 107 disposed on the sides of pad skeleton 104. Pad clips holes 107 may be a hole or receptacle sized and shaped to interact with a corresponding feature of the pump-cap assembly such that the pump-cap assembly may be locked to the pad. Further, pad 102 may include pad opening 109 to allow direct sensing of the wearer's skin by one or more sensors of the pump. For example, the skin sensor(s) and/or the PPG sensor(s) may be positioned at pad opening 109 when the pump is coupled to the pad.

Applicator 100 may include applicator housing 110 and actuator 112. Applicator housing 110 is configured to house the mechanisms for inserting the cannula. After insertion of the cannula, internal component 114 is designed to withdraw and safely store the needle used to pierce the wearer's skin. Actuator 112, upon actuation, causes the cannula to be transcutaneously inserted into the wearer's skin. Actuation of actuator 112 also may unlock applicator 100 from pad 102. Actuation of actuator 112 also may lock the transcutaneously inserted cannula into pad 102. For example, actuation of applicator 100 may insert the cannula transcutaneously, unlock the applicator from the pad, and lock the cannula to the pad in a single actuation. Actuator 112 may release the internal mechanism disposed within applicator housing 110 when actuated by the wearer, thus causing the cannula to advance through the wearer's skin. Actuator 112 may be a button configured to be pressed by the wearer as illustrated, or may be a lever, snap, knob, or the like. The mechanism for inserting the cannula may include internal component 114, biasing member 116, and links 118 and 120, which are disposed within applicator housing 110, and are configured to advance cannula 200 through pad 102 and into the wearer's skin. The mechanism may further include applicator needle 150, which is configured to be disposed within cannula 200 during insertion and withdrawn from cannula 200 after insertion. Self-sealing septum 224 may be disposed within the cannula head of cannula 200 in order to support and guide applicator needle 150 and minimize backflow out of cannula 200.

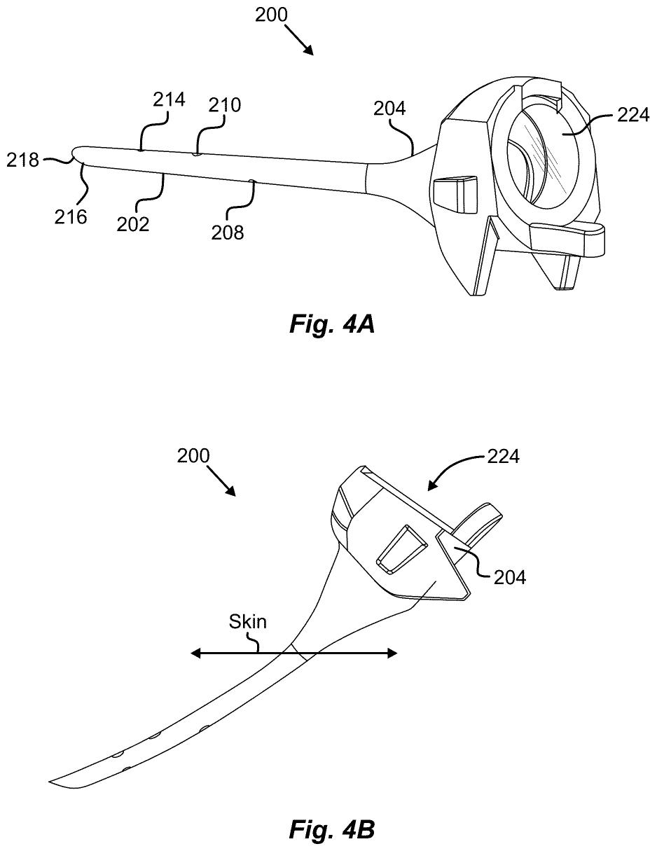

Referring now to FIGS. 4A and 4B, an exemplary cannula for delivering medication is described. Cannula 200 may be injection molded from a single piece of material, which is preferable to extrusion in order to reduce the risk of kinking of cannula 200. Cannula 200 preferably is made from a material that is insulin compatible and flexible and includes cannula head 204 including a septum 224 therein, cannula tip 218, and elongated shaft 202. Elongated shaft 202 is designed to be straight prior to deployment (FIG. 4A) and to curve, responsive to curving of the needle disposed therein, when inserted in the skin (FIG. 4B). The cross-section of cannula 200 may have an oval section shape along the extended shaft for better curve. In this manner, the cannula is thinner in the direction of the curve. Cannula head 204 is disposed at the proximal end of cannula 200 and configured to interact with the applicator needle and the needle through which the medication is delivered. Cannula tip 218 is disposed at the distal end of cannula 200 and may include distal aperture 216 for delivering medication. Elongated shaft 202 may extend between cannula head 204 and cannula tip 218 and may include one or more apertures 208, 210, 212, 214 for delivery of medication. Elongated shaft 202 may increase in diameter towards cannula head 204 and the wearer's skin surface, to mitigate the risk that the delivered medication travels proximally along the outside surface of the cannula to the dermal layer or the surface of the skin. This conical shape may also reduce the risk of kinking of cannula 200.

Following insertion of the cannula 200 with the insertion needle 150, the cannula remains transcutaneously implanted within the wearer's skin for delivery medicament from pump 400. It is therefore important that the applicator is configured such that the cannula 200 is inserted to a depth that provides for adequate insulin absorption. However, there are competing factors in determining how far the cannula should be inserted into the user's body. While a longer cannula would provide a larger volume of fatty tissue for insulin absorption leading to a longer wear time, inserting the cannula too far into the body into muscle causes pain for users. One option that has been employed to balance these factors is inserting the cannula at angle such as that depicted in FIG. 3B, which allows for a longer cannula to be used without inserting the cannula the full length of the cannula depth into the body.

For most users, the depth of the infusion holes of the cannula should be between about 3 mm and 6 mm below the surface of the skin. However, for users with less fat, pain may be experienced with as allow as a 4 mm insertion. Reliable control of insertion depth in a narrow range between 3 mm and 4 mm would therefore be beneficial.

As noted above with respect to FIGS. 4A-4B, one option developed for accommodating these needs was to cause the cannula shaft 202 to be inserted into the body in a curved manner. This would allow for a longer length of cannula to be in the skin for greater infusion capability while limiting the depth to which the cannula is inserted. However, it has been found that it is difficult to reliably insert a curved cannula to an appropriate distance in the skin. One reason for this issue is that a skin surface deformation occurs during cannula insertion, which can affect the curvature of the cannula and the depth of insertion. Embodiments disclosed herein accommodate this skin surface deformation by more reliably controlling the geometry of the skin during insertion rather than the geometry of the cannula to obtain an appropriate depth and angle of insertion.

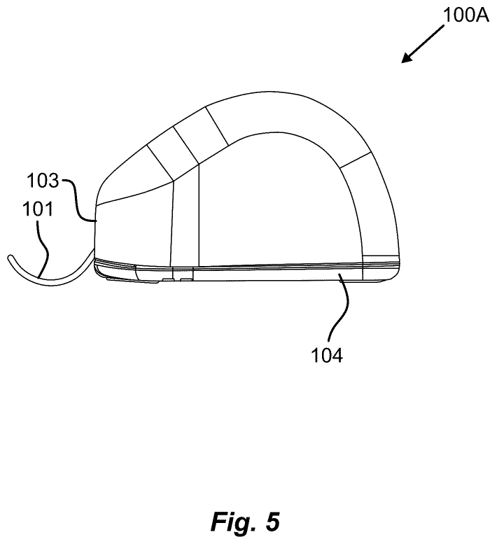

FIG. 5 schematically depicts an applicator 100A for a patch pump system according to the disclosure. Applicator 100A can include similar components to those depicted in FIGS. 3A-3B and is configured to insert a cannula and attach a pad skeleton 104 to the user upon actuation. Applicator 100A further includes a skin deformation projection or bulge 101 at a proximal end 103 of the housing of applicator. Proximal end 103 of applicator is the end nearest to where the cannula is inserted and end towards which the cannula is angled. When the applicator 100A is placed on the user's skin prior to cannula insertion, following removal of the adhesive backing on the underside of the pad skeleton 104, the skin deformation projection 101 compresses and deforms the user's skin near the insertion point of the cannula prior to and during device actuation. This has been found to provide for a better depth and angle of insertion for the cannula with the insertion needle. In various embodiments, skin deformation projection 101 can be formed monolithically with the housing of the applicator to which it is attached or can be a separate selectively attachable accessory that can be employed as desired by a user.

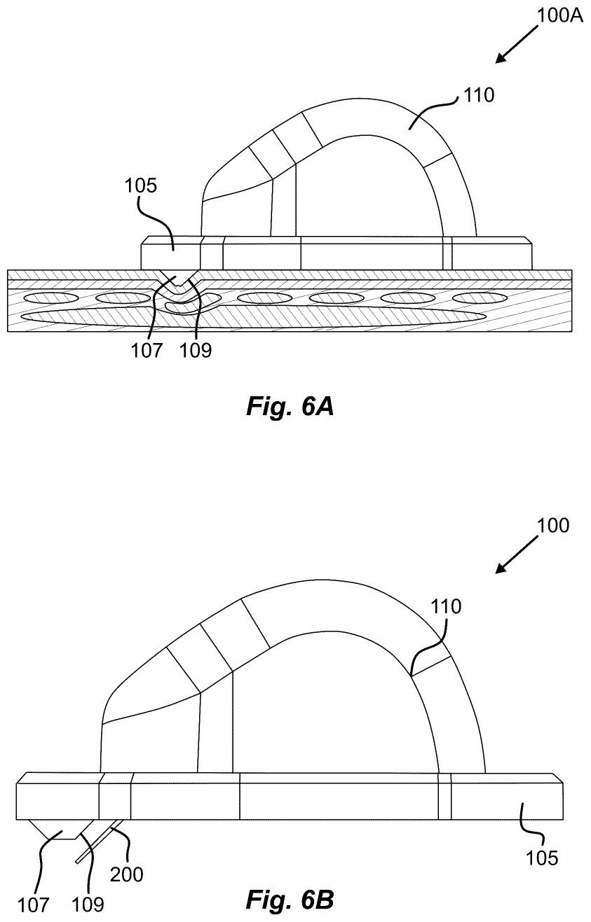

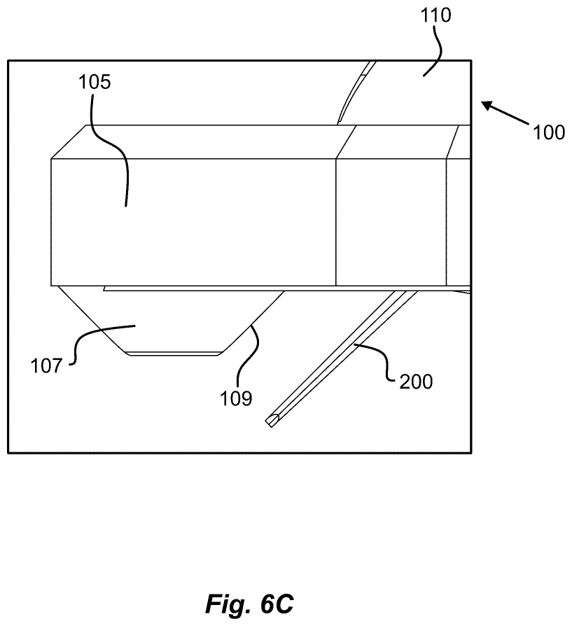

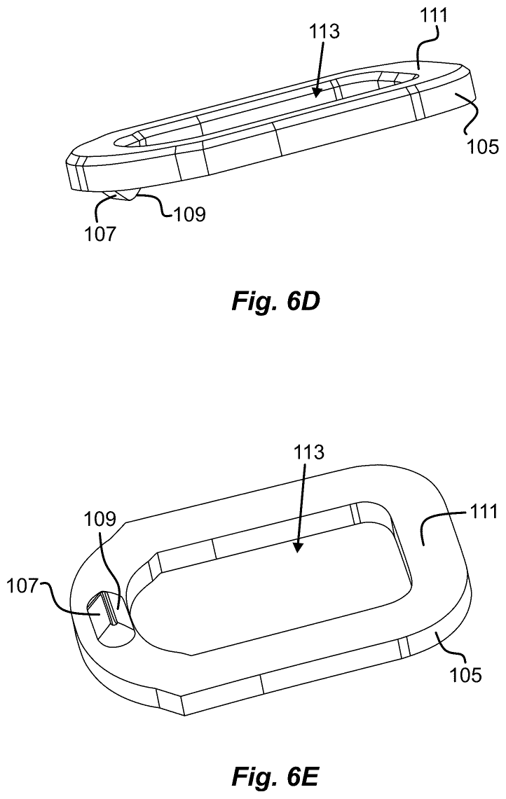

FIGS. 6A-6E schematically depict an applicator 100 having an applicator extension 105 for a patch pump system according to the disclosure. In this embodiment, applicator 100 is not modified and an additionally accessory is employed to provide the effect discussed above. This accessory can be an applicator extension 105 that extends around a perimeter of the main housing 110 of the applicator. Applicator extension 105 includes a body 111 having a cutout or opening 113 sized and shaped to conform to an outer perimeter of applicator 100 and a skin deformation projection or bulge 107 on an underside of body 111. As can be seen in FIG. 6A, when applicator 100B is placed on the user's skin, skin deformation projection 107 causes the skin to deform around the projection and maintains that position while the cannula 200 is inserted. As shown in FIGS. 6B-6C, in embodiments an angled surface 109 of the skin deformation projection 107 may be configured to have the same angle relative to the applicator 100 as the cannula. Although applicator 100 is generally designed for single use, applicator accessory 105 can be reused with a new applicator each time a cannula is being inserted. In a further embodiment, rather than using a separate accessory, a skin deformation projection 107 could initially be contained within the housing of the applicator and an actuation mechanism provided to cause the projection to extend out of the housing and deform the skin when the cannula is going to be inserted.

A study was conducted by inserting an angled cannula into pork belly using an applicator having a skin deformation projection similar to that as depicted in FIGS. 6A-6E (the “controlled” results) and an applicator without such a projection (the “uncontrolled” results). Table 1 below presents the results of measuring 1) the minimum distance between the proximal infusion hole (i.e., infusion hole 208 in FIG. 4A) and the skin surface (the “proximal hole depth”) and 2) the minimum distance between the distal tip 218 of the cannula and the skin surface (the “cannula max depth”).

TABLE 1

| | Proximal hole | Cannula max |
|---|---|---|
| Sample | depth (mm) | depth (mm) |
| Uncontrolled 1 | 1.8 | 5.2 |
| Uncontrolled 2 | 2.6 | 5.1 |
| Uncontrolled 3 | 2.3 | 5.7 |
| Controlled 1 | 1.4 | 3.9 |
| Controlled 2 | 1.5 | 3.4 |
| Controlled 3 | 1.6 | 4 |
| Average uncontrolled | 2.2 | 5.3 |
| Average controlled | 1.5 | 3.8 |
| Average 300/300 test | — | 6.48 |

As can be seen in the table above, the “controlled” results provided by employing an applicator that deforms the skin for insertion of the cannula results in a greater decrease in maximum depth than in the proximal hole depth. Such an approach therefore reduces the risk of pain from inserting the cannula too far into the skin (cannula max depth) without proportionally increasing the possibility of the proximal hole not being inserted far enough (proximal hole depth) for effective infusion absorption. (It is noted that while the proximal hole depth does not reach the desired 3 mm depth, that these results are due to the high skin hardness of pork.)

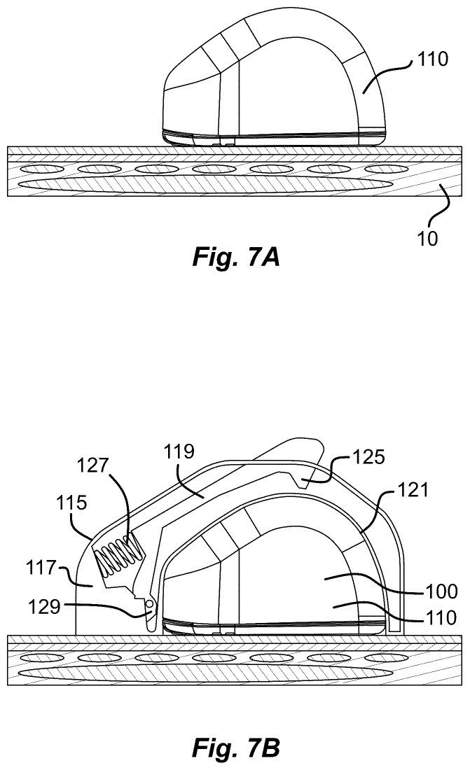

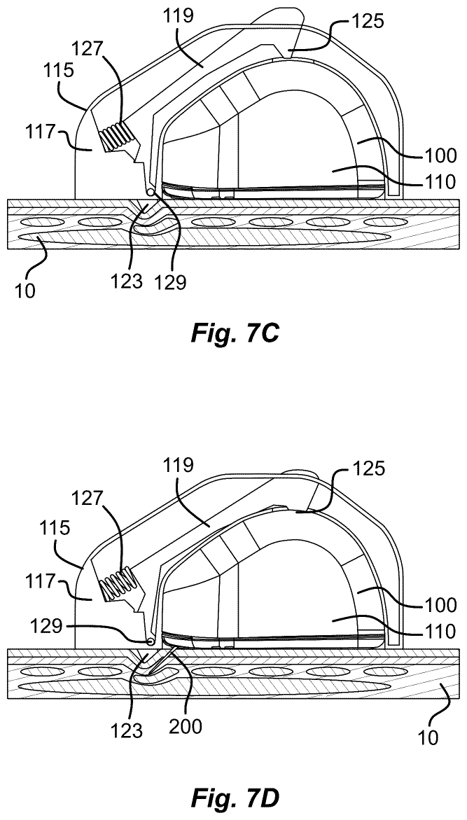

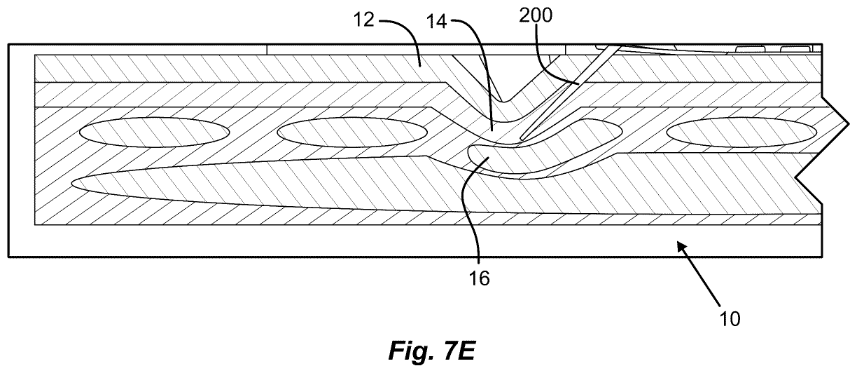

FIGS. 7A-7E schematically depict use of an applicator 100 for a patch pump system having another applicator extension 115 according to the disclosure. FIG. 7A depicts the applicator 100 initially placed on the user's body 100. Next, as shown in FIG. 7B, the applicator extension 115 is placed around the applicator. Applicator extension 115 includes a hull 117 with a movable arm 119 attached thereto. Hull 117 includes a cutout or opening 121 shaped to conform to the outer housing 110 of applicator. Arm 119 includes a skin deformation projection 123 (located within hull 117 as shown in FIGS. 7C-7D) and an applicator actuation projection 125. A spring 127 is positioned between movable arm 119 and a surface of hull 117. Referring now to FIG. 7C, when the movable arm 119 is pushed it causes the spring 127 to compress and skin deformation projection 123 to move downwardly. A depth limiting projection 129 can contact the end of a slot on hull 117 to limit an amount that the skin deformation projection 123 can move. Applicator actuation projection 125 also moves downwardly adjacent to the actuator on the top of the housing 110 of the applicator. The user can then press on the movable arm 119 to actuate the applicator 100 to insert the cannula 200 into the body 10 of the user as shown in FIG. 7D. As can be seen in FIG. 7E, by deforming the skin on insertion, the cannula across the epiderma 12 into the hypoderm 14 without penetrating muscle 16. Following actuation, upon the user releasing pressure on the arm 119 the spring 127 can automatically cause the arm 119 to return to its initially position. In some embodiments, the user pressing on the arm can first cause the skin deformation projection 123 to deform the skin followed by additional pressure to move the applicator actuation projection 125 to actuate the applicator. Alternatively, the skin deformation projection 123 and applicator actuation projection can be actuated generally simultaneously.

In an embodiment, applicator for inserting a cannula into a body of a patient can include a cannula, an insertion needle having a sharpened distal tip to insert the cannula into the body of the patient, an actuation assembly configured to actuate the insertion needle to insert the cannula and a skin deformation projection configured to deform the skin in an area where the cannula is inserted.

In some embodiments, the applicator further includes an applicator housing containing the actuation assembly and the skin deformation projection extends from the applicator housing.

In some embodiments, the skin deformation projection extends from an end of the applicator housing nearest to the area where the cannula is inserted.

In some embodiments, the applicator further includes an applicator housing containing the actuation assembly and an applicator extension including the skin deformation projection.

In some embodiments, the applicator extension comprises a body having an opening configured to receive the applicator housing therein.

In some embodiments, the skin deformation projection extends from an underside of the body of the applicator extension.

In some embodiments, the skin deformation projection comprises an angled surface having a same angle relative to the applicator housing as the cannula following insertion of the cannula into the body of the patient.

In some embodiments, the applicator extension comprises a hull configured to be placed over the applicator housing.

In some embodiments, the applicator extension further comprises a movable arm including the skin deformation projection and an applicator actuation projection.

In some embodiments, force on the arm towards the body of the patient causes both the skin deformation projection to deform the skin and the applicator actuation projection to actuate the actuation assembly to insert the cannula into the body of the user.

In some embodiments, a first force on the arm causes the skin deformation projection to deform the skin and a second subsequent forces causes the applicator actuation projection to actuate the applicator.

In embodiments, an applicator extension for use with an applicator for inserting a cannula into a body of a patient can include a body having an opening configured to receive an applicator housing of the applicator therein and a skin deformation projection extending from the body configured to deform skin of a patient in an area where the cannula is inserted.

In some embodiments, the opening is a cutout through an underside of the body.

In some embodiments, the skin deformation projection extends from the underside of the body of the applicator extension.

In some embodiments, the skin deformation projection comprises an angled surface having a same angle relative to the applicator housing as the cannula following insertion of the cannula into the body of the patient.

In some embodiments, the body comprises a hull configured to be placed over the applicator housing.

In some embodiments, the applicator extension further includes a movable arm including the skin deformation projection and an applicator actuation projection.

In some embodiments, force on the arm towards the body of the patient causes both the skin deformation projection to deform the skin and the applicator actuation projection to actuate the applicator to insert the cannula into the body of the user.

In some embodiments, a first force on the arm causes the skin deformation projection to deform the skin and a second subsequent forces causes the applicator actuation projection to actuate the applicator.

In some embodiments, the applicator extension further includes a spring configured to cause the arm to return to an original position when the force is release

Although embodiments described herein may be discussed in the context of the controlled delivery of insulin, delivery of other medicaments, singly or in combination with one another or with insulin, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, treatment of various conditions including, e.g., pulmonary hypertension, or any other suitable indication or application. Non-medical applications are also contemplated.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

1. An applicator for inserting a cannula into a body of a patient, comprising:
a cannula;
an insertion needle having a sharpened distal tip to insert the cannula into the body of the patient;
an actuation assembly configured to actuate the insertion needle to insert the cannula; and
a skin deformation projection configured to deform the skin in an area where the cannula is inserted.

2. The applicator of claim 1, further comprising an applicator housing containing the actuation assembly and wherein the skin deformation projection extends from the applicator housing.

3. The applicator of claim 2, wherein the skin deformation projection extends from an end of the applicator housing nearest to the area where the cannula is inserted.

4. The applicator of claim of claim 1, further comprising an applicator housing containing the actuation assembly and an applicator extension including the skin deformation projection.

5. The applicator of claim 4, wherein the applicator extension comprises a body having an opening configured to receive the applicator housing therein.

6. The applicator of claim 5, wherein the skin deformation projection extends from an underside of the body of the applicator extension.

7. The applicator of claim 4, wherein the skin deformation projection comprises an angled surface having a same angle relative to the applicator housing as the cannula following insertion of the cannula into the body of the patient.

8. The applicator of claim 4, wherein the applicator extension comprises a hull configured to be placed over the applicator housing.

9. The applicator of claim 8, wherein the applicator extension further comprises a movable arm including the skin deformation projection and an applicator actuation projection.

10. The applicator of claim 9, wherein force on the arm towards the body of the patient causes both the skin deformation projection to deform the skin and the applicator actuation projection to actuate the actuation assembly to insert the cannula into the body of the user.

11. The applicator extension of claim 10, wherein a first force on the arm causes the skin deformation projection to deform the skin and a second subsequent forces causes the applicator actuation projection to actuate the applicator.

12. An applicator extension for use with an applicator for inserting a cannula into a body of a patient, comprising:
a body having an opening configured to receive an applicator housing of the applicator therein; and
a skin deformation projection extending from the body configured to deform skin of a patient in an area where the cannula is inserted.

13. The applicator extension of claim 12, wherein the opening is a cutout through an underside of the body.

14. The applicator extension of claim 13, wherein the skin deformation projection extends from the underside of the body of the applicator extension.

15. The applicator extension of claim 12, wherein the skin deformation projection comprises an angled surface having a same angle relative to the applicator housing as the cannula following insertion of the cannula into the body of the patient.

16. The applicator extension of claim 12, wherein the body comprises a hull configured to be placed over the applicator housing.

17. The applicator extension of claim 16, further comprising a movable arm including the skin deformation projection and an applicator actuation projection.

18. The applicator extension of claim 17, wherein force on the arm towards the body of the patient causes both the skin deformation projection to deform the skin and the applicator actuation projection to actuate the applicator to insert the cannula into the body of the user.

19. The applicator extension of claim 18, wherein a first force on the arm causes the skin deformation projection to deform the skin and a second subsequent forces causes the applicator actuation projection to actuate the applicator.

20. The applicator extension of claim 17, further comprising a spring configured to cause the arm to return to an original position when the force is released.
